(19) United States
(12) Patent Application Publication
(10) Pub. No.: US 2025/0032073 A1
(43) Pub. Date: Jan. 30, 2025

(54) OPERATING METHOD AND SYSTEM FOR AN X-RAY FACILITY FOR CREATING RECONSTRUCTED IMAGES OF A BODY REGION

(71) Applicant: Siemens Healthineers AG, Forchheim (DE)

(72) Inventors: Matthias LICHY, Nuernberg (DE); Bernd HOFMANN, Erlangen (DE); Ralf GUTJAHR, Nuernberg (DE); Bernhard SCHMIDT, Fuerth (DE)

(73) Assignee: Siemens Healthineers AG, Forchheim (DE)

(21) Appl. No.: 18/783,973

(22) Filed: Jul. 25, 2024

(30) Foreign Application Priority Data
Jul. 28, 2023 (DE) 10 2023 207 229.7

(51) Int. Cl. A61B 6/00; A61B 6/03; A61B 6/50

(52) U.S. Cl. CPC A61B 6/481; A61B 6/032; A61B 6/482; A61B 6/504

(57) ABSTRACT

An operating method comprises: triggering administration of a first contrast medium application for presaturation of the body region with contrast medium, wherein the first contrast medium application is administered to achieve a desired equilibrium concentration; triggering administration of a second contrast medium application in the form of a bolus larger than in the first contrast medium application; recording spectral image data at at least two different beam energies; reconstructing images from the spectral image data; and outputting the images.

### CROSS-REFERENCE TO RELATED APPLICATION(S)

The present application claims priority under 35 U.S.C. § 119 to German Patent Application No. 10 2023 207 229.7, filed Jul. 28, 2023, the entire contents of which is incorporated herein by reference.

### FIELD

One or more embodiments of the present invention comprise an operating method and a system for an X-ray device and/or facility for creating reconstructed images of a body region, in particular of CT angiographies, as well as a control device and/or facility and a computed tomography system. One or more embodiments of the present invention relate in particular to a selective vascular and parenchyma representation by controlling a concentration threshold value-based contrast medium application.

### BACKGROUND

Angiography is a method established in medicine for the radiological representation of vessels, usually blood vessels, via diagnostic imaging methods. For this purpose, for the evaluation of vessels and parenchyma, a contrast medium is usually injected into the vessels, typically in arteries, and at the same time an image is taken so that the vessel becomes visible in the X-ray image. The recordings can be X-ray recordings, CT recordings (CT: “computed tomography”) or MRI recordings, wherein one or more embodiments of the present invention relate primarily to X-ray recordings, in particular CT recordings.

Often, the contrast medium is injected after (previous) native recordings. Depending on the region, organ, pathology and/or problem, timed images are created and, if necessary, repeated.

In the specific example of aortic dissection, an arterial phase is recorded. In the presence of inserted endoprostheses, leaks or larger thrombi or false lumina, additional recordings must be made. The representation of the inlet and outlet of false vascular lumina is of great importance for therapy. However, they are often not or only insufficiently recorded in the initial recording. The amount of contrast that is applied is optimized for the representation of a normally perfused lumen, both in terms of timing (scan start and scan duration) and quantity (for example scaled to patient weight, -BMI, -BSA, -LBW). As a result, it is possible for the problem to occur, in particular in emergency situations, that acute bleeding into existing bleeding cavities can be differentiated poorly. Even with optimal timing of the bolus of contrast medium administration, pathology can also lead to massively changed circulation times in the vascular section of interest, which can lead to poorer or even a lack of contrast. Ultimately, misinterpretations of the findings by the operating personnel and omission of further scans under the above conditions can also lead to problems.

These problems can occur in a number of other regions and problems, such as for instance due to arteriovenous malformations of the brain, the lung or the liver, in addition to hyper vascularized tumors, for example in hepatocellular carcinoma or in neuroendocrine tumors, etc., and also temporally in hypovascularized tumors, for example pituitary adenomas.

### SUMMARY

Up to now, the above-discussed problems have been counteracted by performing further scans or examinations (one after the other or separated in time), which, however, leads to increased radiation exposure of the patient. In addition, each additional examination blocks the imaging system used, which could otherwise be used for other examinations. In some cases, further doses of contrast medium are also given if the indication has been made or the incorrect implementation has been detected in a timely manner. In the case of iodized contrast medium, this can lead to an increased risk of acute kidney damage or contrast-induced nephropathy.

It is an object of one or more embodiments of the present invention to provide an alternative, more comfortable method and a corresponding system for creating recordings or images, in particular CT angiographies, in particular for controlling a CT system, with which the disadvantages described above are avoided.

At least this object is achieved by a method, a system, a control facility (also referred to herein as a control device) and an X-ray facility (also referred to herein as an X-ray device or system) as claimed.

In accordance with an embodiment of the present invention, an operating method is proposed for an X-ray facility, for example a CT scanner, for creating reconstructed images of a body region, comprising the steps:
triggering an administration of a first contrast medium application for presaturation of the body region with contrast medium, in particular until a desired equilibrium concentration is achieved,
triggering an administration of a second contrast medium application in the form of a bolus larger than in the first contrast medium application,
recording spectral image data (D) at at least two different beam energies,
reconstructing images (Z, E) from the spectral image data (D),
outputting the reconstructed images (Z, E).

Embodiments of the present invention are based on a combination of a novel contrast medium protocol with the recording of spectral (or spectrally resolved) image data and, in particular, in addition on the possibility of an image reconstruction that depends on the concentration of the contrast medium, as described in more detail below. A particularly preferred contrast medium is iodine.

In angiography images, in particular also in CT angiographies, images of a body region are recorded in which the vessels and in particular also the surrounding tissue are displayed. The vessels are provided with a contrast medium by the contrast medium application, so that they can be better distinguished from the surrounding tissue, and are recorded using a CT system.

Overall, in accordance with an embodiment of the present invention, two contrast medium applications are performed. The first contrast medium application serves to presaturate the body region. In this case, a comparatively smaller amount of contrast medium or a contrast medium administration with a comparatively lower contrast medium concentration is applied in the first contrast medium application. In the second contrast medium application, a comparatively higher amount of contrast medium or a contrast medium administration with a comparatively higher contrast medium concentration is applied. For this purpose, for example, as part of the first contrast medium application, so much contrast medium is administered until in particular a desired equilibrium concentration is achieved. The equilibrium concentration corresponds to a uniform distribution of the contrast medium (for example the iodine content) over the entire blood volume in the patient or at least in the body region to be recorded. The equilibrium concentration is known from the prior art. The distribution is uniform, for example, if a state has been established in the tissue in which the contrast medium of the bolus has been distributed. In principle, it can be said that a uniform distribution has resulted in an area when the concentration of the contrast medium in this area has reached its maximum or decreases again. For example, the same contrast medium concentration should prevail everywhere in an organ. However, it is possible that contrast medium accumulates in places in an organ due to pathologies or for biological/geometric reasons. It is nevertheless clearly visible when a uniform distribution has been achieved. For this purpose, it should be noted that it is not necessary to always determine this again. There are already studies that indicate after which time period an equilibrium concentration is achieved after a contrast medium application at a given contrast medium concentration or amount in a body region.

The first contrast medium application therefore presaturates the tissue of interest up to a desired concentration, in particular a (for example patient weight-specific) equilibrium concentration is achieved. In contrast to conventional imaging with contrast medium having a desired concentration for the first contrast medium application, a significantly reduced contrast is preferably meant. This is based in particular on the contrast/noise ratio (CNR), which is important for the diagnosis in the low keV value range.

The contrast medium is preferably applied by an application unit, for example a controlled injector, which is suitable for diluting a contrast medium concentration for an application.

The equilibrium concentration depends on the body region that is examined and, if necessary, also on the type of examination. In this regard, it can be assumed that less than 90%, in particular less than 50%, but preferably more than 10%, in particular more than 30%, is administered for an examination of a body region in the context of the first contrast medium application, compared with a conventional contrast medium application.

The phase of the first contrast medium application can also be used to determine an optimal point in time for the second contrast medium application. This can be performed, for example, by analogy with a test bolus method. A test bolus method of this type is disclosed in DE 10 2006 055 167 A1, for instance. It is noted that it can be advantageous to perform a (pre-) CT scan between the first contrast medium application and the second contrast medium application, for example to record the vessels in the state of the equilibrium concentration or arterial phases.

In the context of the second contrast medium application, a highly concentrated, as sharply defined bolus (in other words, a short injection time with a high flow rate) is now administered, possibly adapted to the cardiac output and the anatomy or pathology to be analyzed. The type of examination and the body area should also be taken into account.

In the second contrast medium application, however, the bolus is always larger than in the first contrast medium application, in particular in the bolus of the second contrast medium application, the contrast medium amount or the contrast medium concentration is higher than in the first contrast medium application.

In this bolus of the second contrast medium application, the contrast medium is preferably administered at more than 4 ml/s, preferably more than 6 ml/s or even more than 7 ml/s, for example at 8 ml/s.

In comparison with the first contrast medium application, the second contrast medium application should in particular have a higher flow rate, in particular more than 2 times higher, preferably more than 4 times higher. However, this can depend on the anatomy. The second contrast medium application should have a shorter injection time than the first contrast medium application and in particular should take place in less than half the time, preferably in less than 25% of the time. It is however often bound to the limits of the method of administration or to flow maxima. Here it should be noted that the injection time should not be shorter than a limitation of the application unit and no more contrast medium should be injected per unit time than is medically sensible, since at high flow rates the risk of extravasation or paravasation increases. A minimum injection duration can be determined by the necessary volume, which in turn usually depends on the patient's weight.

In most cases, a contrast medium is applied through the right arm vein in the area of the antecubital fossa, less often into the left arm vein. Even less often, other veins in arms or legs are used and even less often an application of contrast medium takes place via a peripheral central catheter, into the jugular vein.

After the two contrast medium applications have been performed, spectral image data, for example CT data, is now recorded at at least two different beam energies or at at least two different beam spectra. This is preferably done by using X-rays with at least two different spectra (for example dual source imaging, kV switching, dual spiral CT) or by resolving a spectrum (for example energy-resolving dual layer detectors, energy-resolving photon counting detectors, split filter arrangements). The recording point in time and the number of recordings are based on the technique used. For example, a single recording is sufficient in the case of dual layer detectors, photon counting detectors or split filter arrangements.

Depending on the type of examination, for example early arterial phase, arterial phase, venous phase, portal venous phase, late phase, the images, for example CT scans, can be delayed. In the arterial phases in particular, the ideal point in time for a scan depends on patient-specific parameters (for example cardiac performance).

After the recording, images are reconstructed from the recorded image data, wherein in particular two or more images of the same region are generated in which different image portions have been highlighted differently. These images are then preferably offset with each other by special calculation methods, so that areas are highlighted and other areas are attenuated. Such computing methods are known, act on the values of the pixels and comprise, for example, one of the basic arithmetic types, in particular an addition or subtraction or one of the known image processing techniques for offsetting two image planes. Particularly preferably, an arterial image is extracted, a so-called “delayed” image is extracted or a quantization of the contrast medium is performed. An arterial image can be simulated, for example, on a native image. It is also possible to calculate a predetermined contrast medium concentration from an image, for example a concentration below a threshold value or outside a predetermined value range. The reconstructed images are then output.

With the special feature in the case of spectral image data, for example in the case of spectral CT (different image information for the same regions on the basis of the recorded spectra), it is possible to generate concentration-dependent masks for the further image processing that is addressed. Particularly preferably, a predetermined contrast medium concentration, in particular the equilibrium concentration, is calculated from an image.

A system in accordance with an embodiment of the present invention, for implementing an operating method for an X-ray facility for creating reconstructed images of a body region is preferably designed so as to implement a method, in accordance with an embodiment of the present invention, and comprises the following components:
an application unit (14) that is designed so as to trigger an administration of a first contrast medium application for presaturation of the body region with contrast medium, in particular until a desired equilibrium concentration is achieved, and for triggering an administration of a second contrast medium application in the form of a bolus larger than in the first contrast medium application, wherein in this bolus the contrast medium is administered at more than 4 ml/s,
a number of X-ray facility components (2), for example a CT scanner, that are designed so as to record spectral image data (D) at at least two different beam energies,
a reconstruction unit (13) that is designed so as to reconstruct images (Z, E) from the image data (D), preferably wherein, in the reconstruction of the images (Z, E), concentration-dependent portions in the image (Z, E) are selectively enhanced, attenuated or masked,
an output unit (12) that is designed so as to output the reconstructed images (Z, E).

The X-ray facility is in particular a CT scanner.

The mode of operation of the components of the X-ray facility has already been described by way of example in the context of the method. The application unit is preferably designed so as to automatically dilute a contrast medium for the first contrast medium application to or by a first defined value and to apply the same in a diluted manner, and to dilute the second contrast medium application to or by a second defined value and to apply the same in a diluted manner, in particular preferably an undiluted manner. Thus, only one storage container for the contrast medium and one for the solvent, for example saline solution, is required. It can also be preferred that the contrast medium is diluted for both contrast medium applications, but to a lesser extent for the second contrast medium application than for the first contrast medium application.

A control facility in accordance with an embodiment of the present invention, for controlling an X-ray facility, in particular a computed tomography system, is designed so as to implement a method in accordance with an embodiment of the present invention and/or comprises a system in accordance with an embodiment of the present invention.

An X-ray facility in accordance with an embodiment of the present invention, in particular a computed tomography system, comprises a system in accordance with an embodiment of the present invention and/or a control facility in accordance with an embodiment of the present invention.

A large proportion of the above-mentioned components of the system can be realized entirely or in part in the form of software modules in a processor of a corresponding computer system, for example of a control facility of a computed tomography system. A largely software-based realization has the advantage that computer systems that are already previously used can be retrofitted in a simple manner by a software update in order to function in the manner in accordance with an embodiment of the present invention. In this respect, the object is also achieved by a corresponding computer program product having a computer program, which can be loaded directly into a computer system, having program sections in order to perform the steps of the method in accordance with an embodiment of the present invention, at least the steps that can be performed by a computer, if the program is executed in the computer system. It should be noted that the recording of spectral image data in this implementation corresponds to the reception of image data, for example by a data bus or by reading a storage unit. Such a computer program product in addition to the computer program can, where necessary, comprise additional elements such as for example a documentation and/or additional components, even hardware components such as for example hardware keys (dongles etc.) in order to use the software.

For the transport to the computer system or to the control facility and/or to the storage device at or in the computer system or the control facility, it is possible to use a computer-readable medium, for example a memory stick, a hard drive or another transportable or fixedly installed data carrier on which are stored the program sections of the computer program, which can be read and executed by a computer system. The computer system can have for example for this purpose one or multiple cooperating microprocessors or the like.

It should be noted that method steps such as triggering the administration of contrast medium and recording image data correspond to the output of corresponding control commands.

Further, particularly advantageous embodiments and developments of the present invention are provided in the claims and also the subsequent description, wherein the claims of a claim category can also be developed in a similar manner to the claims and description parts with regard to another claim category and in particular also individual features of different exemplary embodiments or variants can be combined to form new exemplary embodiments or variants.

In accordance with a preferred method, segmentation of different base materials takes place in the reconstruction of the images. Depending on the concentration, segments in the image are selectively highlighted, attenuated or masked. This can be performed depending on the problem and anatomy. In a simple case, segmentation can take place in that all pixels that lie within a certain value range are allocated to a base material. However, it is advantageous to logically link images of different recording energies, for example, the image value in the one image must lie in a first value range and in the second image in another value range.

In the example of aortic dissection with entry into the wrong lumen and smaller covered bleeding, the following postprocessing steps could be used:

In the case of a problem with regard to a covered rupture versus a persistent bleeding, a simple recognition of the contrast medium (in particular iodine) in the bleeding area for the visualization of a potential jet takes place, for example, by analogy with already existing problems in the brain. In this case, it is possible to take into consideration a selective enhancement of areas having an increased contrast medium concentration. In this case, in contrast to conventional CT, the concentration-dependent representation would represent both the extent of the local contrast medium outlet and the localization.

In the case of a representation of the arterial lumen of the aorta, high contrast medium concentrations (for example, iodine concentrations) are visualized on virtual native images for the representation of the true lumen of the aorta. In this case, the increase in density is eliminated by the contrast medium in the, for example, venous vascular system. By analogy with the above, this method can also be used, for example, to visualize a jet of higher contrast medium concentrations in the wrong lumen.

In the case of a visualization of the wrong lumen and differentiation of thrombi, the selective enhancement of the contrast for low concentrations takes place in order to represent the extent of the perfusion of the wrong lumen. In addition, further information from the spectral image data can be included. For example, it is possible to detect fresh versus organized thrombi. In this example, in accordance with the optimal visualization areas with a high concentration can be masked, not further amplified or can be linearly co-amplified. Analogously, it is conceivable that a separation of arterial and venous phase in the brain or bronchus takes place or the arterial and portal venous phase of the KM accumulation in the liver is distinguished.

A selective enhancement preferably takes place in areas having increased contrast medium concentrations. The enhancement preferably depends on the phase and the anatomy or on the physiology. In early phases of contrast medium application, the concentrations in the arteries are increased. In the later the areas of the parenchyma. In the liver in particular, the enhancement is significantly increased in later phases, since the liver is supplied with almost ⅔ of portal venous supply, i.e. after a longer transit time.

Contrast medium concentrations, in particular iodine concentrations, are preferably visualized on virtual native images, in particular contrast medium concentrations that lie outside a predetermined value range and in particular are particularly high.

The value range preferably depends on whether a contrast medium concentration in a vessel or in the parenchyma is considered. A high contrast medium concentration in the vessel can correspond, for example, to approximately 10 mgI/ml and a low 0.2 mgI/ml. The values for the kidney (medulla) can scale, for example, with about ⅓ and those in the liver parenchyma and in the pancreas with about 1/10. Contrast medium concentrations are preferably visualized if they are greater than 5 mgI/ml, in particular greater than 8 mgI/ml or even greater than 9 mgI/ml.

A selective enhancement of a contrast preferably takes place for contrast medium concentrations that are below a predetermined threshold value. In this case, further information from the spectral image data is preferably included for this, in particular a detection of fresh versus organized thrombi. The determination of the contrast medium concentration is preferably determined by spectral image data recordings. Preferably, the density of thrombi is also measured and analyzed (ρ/z) or spectrally thrombi types are determined.

Areas with a contrast medium concentration above a predetermined threshold value (in particular in accordance with the optimal visualization) are preferably masked, not further amplified or linearly co-amplified. The threshold value for a contrast medium concentration in an artery is preferably specified by the concentration in the aorta. With regard to later phases, the threshold value can depend on the native values. An optimal visualization is sought, which has a low signal/noise ratio.

A separation of arterial and venous phase in the brain or bronchus preferably takes place. In this case, the separation is preferably based on the proportion of the contrast in the arterial part of the body region compared to the proportion of the contrast in the venous part.

A distinction is preferably made between an arterial and portal venous phase of the accumulation of contrast medium in the liver. The start of the portal venous phase of the liver is preferably indicated by the point in time of the peak enhancement in the spleen. Since, as stated above, the liver is largely supplied with portal venous supply, the rate of enhancement at this point in time is higher than at the arterial phase.

In accordance with a preferred method, a VNC image (VNC: “Virtual non contrast”) and a material image is reconstructed. The VNC image simulates a CT image without contrast medium, i.e. imitates a so-called “true non-contrast” image. The material image represents information of local concentrations of the contrast medium, i.e. it allows conclusions to be drawn about the contrast medium concentration, in particular in mgI/ml. Depending on the phase, the concentrations in arteries/organs/veins are high or low. A result image is then created from these images by image superimposition, in particular by subtraction methods and/or by masking image information that lies outside a predetermined value range. Areas in which more (hyper vascular) or less (hypo vascular) contrast medium is recorded are thus recognizable. This enables information that can be used for differential diagnosis. The attenuation or masking can be controlled via thresholds. These thresholds could, for example, be set manually (for example, masking concentrations below 1 mgI/ml) or automatically (via cluster algorithms, for example, k-means).

In accordance with a preferred method, at least the first contrast medium application is performed using an application unit, in particular comprising an automatically controlled injector. A contrast medium concentration is preferably selected in this case for the second contrast medium application and this contrast medium concentration is diluted for the application for the first contrast medium application. Basically, a contrast medium concentration can be diluted as desired with saline solution. It is particularly advantageous if both contrast medium applications are performed via the same application unit, in particular the same injector, and the first contrast medium application is diluted and the second contrast medium application is undiluted.

In accordance with a preferred method, the first contrast medium application is used for determining the point in time of the second contrast medium application. This can be performed in particular via a test bolus. After application of the test bolus, a sequence of low-dose CT scans, for example, can then be recorded and the local “flood” of contrast medium can be determined (retrospectively) from these. The so-called “time to peak” (ttp), i.e. the time to the maximum of the contrast medium concentration, can be determined in this way.

In accordance with a preferred method, in the second contrast medium application, the contrast medium is administered at more than 4 ml/s, preferably at more than 5 ml/s, in particular at more than 6 ml/s (100 ml, i.e. within 16.6 s). Higher rates of more than 7 ml/s or even more than 8 ml/s (i.e. 100 ml within 12.5 s) are also preferred. The second contrast medium application is preferably adapted to the cardiac output and/or to the anatomy to be analyzed and/or to the pathology. Information about the cardiac performance, i.e. also the cardiac output, can be derived from the above-described measurement of the test bolus. For example, the point in time of optimal enhancement is delayed in persons with reduced cardiac performance. In the case of the test bolus, the timing is determined as described above. However, there are also pathologies that can influence the dynamics. These are, for example, various heart diseases (e.g. shunts or defective heart valves), dissections or aneurysms. The point in time of the scan and/or the volume of the contrast medium are preferably adjusted. The injection site is preferably not adjusted.

The bolus administration in the second contrast medium application is preferably equivalent in behavior to a conventional contrast medium application. In the first contrast medium application to achieve the equilibrium phase, the injection rate is basically insignificant, at least as long as no timing determination is to be performed. In the second contrast medium application, a high flow is important.

The method is preferably combined with information from spectral topograms for planning a CT examination, in particular by automated detection of the pathological aorta with a protocol proposal and placement of the ROI so as to determine the cardiac output so as to determine the second bolus phase or so as to estimate the soft tissue compartment, including determination of fatty tissue to parenchyma ratio, and based on this, determination of the necessary presaturation. When metal or endografts are detected, the reconstruction parameters are preferably adjusted.

In accordance with a preferred method, parameters of the method are selected in dependence upon an expected pathology, in particular a tumor biology, and/or a selected examination type and/or a body region to be examined. These parameters are preferably limits of value ranges and/or threshold values and/or applied contrast medium quantities and/or contrast medium concentrations and/or the reconstruction of images, in particular kernels for reconstruction. As already mentioned above, the contrast medium application, for example, depends strongly on the body region. However, different types of examinations can also require different contrast medium applications. When examining pathologies, different tumor biologies, for example, could also require different contrast medium applications. The same applies to the reconstruction and post-processing of images.

For example, in the case of oncological problems for the representation of various aspects of the tumor, a possibility for manual or automated finer adjustment of the threshold values could be advantageous. An adaptation to the tumor biology with regard to the duration of the presaturation and height is likewise also advantageous. In addition, a particularly high resolution for the representation of the microarchitecture of the vessels is advantageous, in particular for tumor vascularization. In this case, it is preferred to also generate specially adapted kernels and visualization options on pure contrast medium-based data, but the use of concentration-dependent threshold values is also advantageous here. Images that represent a high concentration show, for example, the microarchitecture of malignant tissue, images that represent a normal concentration show normal parenchyma tissue, images that represent a very low concentration show, for example, the diffusion of the contrast medium into a necrosis zone.

A kernel is used in a reconstruction algorithm for CT image data. The kernel determines the sharpness, granularity, contrast resolution and edge information of the reconstructed images. The choice of the kernel depends on the diagnostic problem. One speaks of “sharp” kernels (for example, for fine structures of bones and vessels) and “soft” kernels (for organ imaging). It is preferable that a list of kernels is available for different reconstructions, for example for the reconstruction of images of the areas: Body, vascular, head, quantitative, high-resolution, etc. Different kernels can in particular also have different sharpness levels. A high resolution of vessels can be achieved, for example, by recording modes with low collimation (for example, so-called ultra high resolution” modes) and reconstructions with thin layers, low increments and a sharp kernel.

In accordance with a preferred method, additional recordings of image data are performed between the first contrast medium application and the second contrast medium application and/or at different recording points in time after the second contrast medium application. In particular, recordings of spectral image data are performed at at least two different beam energies or two different beam spectra. Preferably in this case a result image is created for different recording points in time from the respective recorded images by image superimposition, in particular by subtraction methods, and/or by masking image information that lies outside a predetermined value range.

The present invention is once again further illustrated and explained by way of example below with reference to the attached figures with the aid of exemplary embodiments. In this case, identical components are provided with identical reference numerals in the different figures. In general, the figures are not to scale.

In particular, the features and advantages described in connection with the methods in accordance with an embodiment of the present invention can also be embodied as corresponding subunits of the system in accordance with an embodiment of the present invention or of the control facility in accordance with an embodiment of the present invention, of the X-ray facility in accordance with an embodiment of the present invention and of the non-transitory computer program product in accordance with an embodiment of the present invention.

Conversely, the features and advantages described in connection with the system in accordance with an embodiment of the present invention or the control facility in accordance with an embodiment of the present invention, the X-ray facility in accordance with an embodiment of the present invention and the non-transitory computer program product in accordance with an embodiment of the present invention can also be designed as corresponding method steps of the method in accordance with an embodiment of the present invention.

### BRIEF DESCRIPTION OF THE DRAWINGS

In the drawings:

FIG. 1 shows a roughly schematic representation of an X-ray facility with an exemplary embodiment of a control facility having a system in accordance with the present invention for implementing the method,

FIG. 2 shows a sketch of the method sequence.

### DETAILED DESCRIPTION

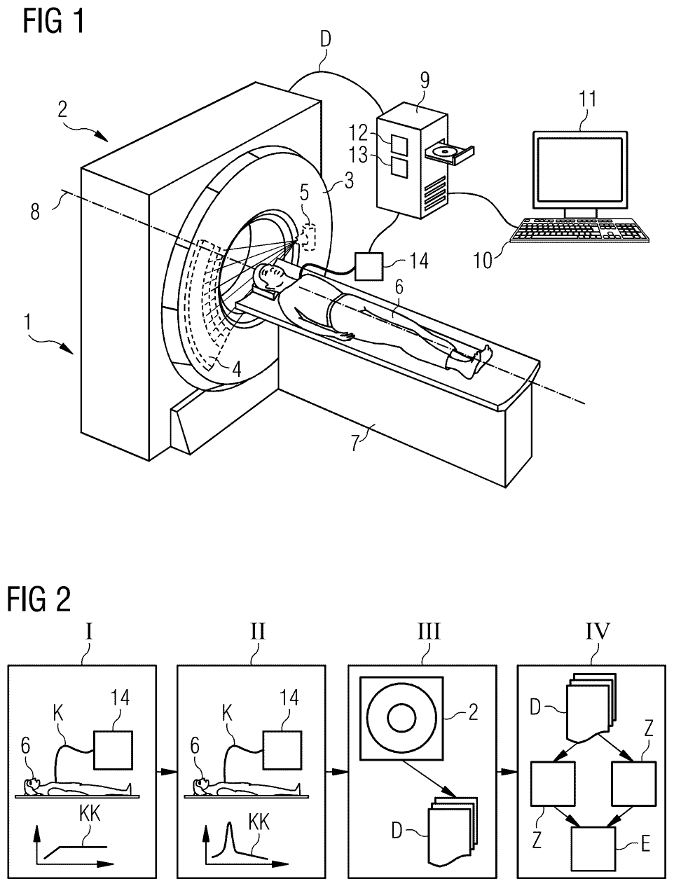

FIG. 1 illustrates an embodiment of an X-ray facility, here in the form of a computed tomography system (CT system) 1 having a radiation detector 4 and a radiation source 5. The radiation source 5 is designed so as to expose the radiation detector 4 to radiation. The illustrated CT system 1 comprises a gantry 2 having a rotor 3. The rotor 3 comprises an X-ray source as radiation source 5 and the radiation detector 4, which is designed to detect X-ray radiation.

The radiation detector 4 and/or the radiation source 5 are designed for spectral CT recordings. For example, the radiation source 5 can be broadband and the radiation detector 4 can detect two or more energies in a resolved manner. However, the radiation source 5 can also emit two or more different spectra in succession, which the radiation detector 4 then detects. It is also possible for the gantry 2 to have two or more systems of radiation source 5 and radiation detector 4 that measure in different energy spectra.

The rotor 3 is rotatable about the axis of rotation 8, which can be regarded here simultaneously as the longitudinal axis 8 of the patient. The patient 6 is mounted on the patient bed 7 and is movable along the axis of rotation 8 through the gantry 2. The computing unit 9 is provided for controlling the imaging system 1 and/or for generating an image data set based on signals that are detected by the radiation detector 4.

A (raw) X-ray image data set of the object 6 is usually recorded from a plurality of angular directions by the radiation detector 4 at one beam energy in each case, that is to say two or more raw data sets. Subsequently, based on the (raw) X-ray image data set, a final image data set can be reconstructed via a mathematical method, for example comprising a filtered back projection or an iterative reconstruction method. In this case, the image data set comprises images Z, E that have been reconstructed using the method in accordance with an embodiment of the present invention.

The computing unit 9 can comprise or be a control facility 9 for controlling the CT system 1 and a generation unit for generating an X-ray image data set. In the case shown here, the computing unit 9 is the control facility 9.

Furthermore, an input facility 10 and an output facility 11 are connected to the computing unit 9. The input facility 10 and the output facility 11 can, for example, enable an interaction by a user or the representation of a generated image data set or output a determined solution to the problem.

In this case, the computing unit 9 as a control facility 9 comprises a data interface 12 for receiving CT data D of a recording and a reconstruction unit 13 for reconstructing images Z, E in accordance with an embodiment of the present invention (see, for example, FIG. 2). The computing unit is also designed so as to control an application unit 14 in accordance with the method in accordance with an embodiment of the present invention.

The data interface 12 is designed so as to receive spectral CT data D at at least two different mean beam energies and so as to output the reconstructed images E. The data interface is also used here as an output unit 12.

The reconstruction unit 13 is designed so as to reconstruct at least intermediate images Z or result images E from the CT data D. It can be designed in such a manner that it reconstructs intermediate images Z from CT data D and then calculates a number of result images E from these intermediate images Z, but it can also reconstruct a number of result images at the same time based on a certain consideration of the CT data D.

The application unit 14, the reconstruction unit 13 and the CT components that are necessary for recording images, for example the gantry 2, here form an example of the system in accordance with an embodiment of the present invention. In principle, the CT system 1 can also be regarded as a system in accordance with an embodiment of the present invention here, since all components are already integrated there.

The CT system shown here can be, for example, a CT system 1 having photon-counting detectors, a dual source CT system 1, a CT system 1 having a dual layer detector, a CT system 1 having kV switching or a CT system 1 having divided prefilters.

FIG. 2 illustrates a block diagram of a preferred exemplary embodiment of a method in accordance with the present invention for creating CT angiographies E of a body region.

In step I, the body region is presaturated with a first contrast medium application until a desired equilibrium concentration is achieved. This equilibrium concentration is present, for example, if the contrast medium K has been uniformly distributed in the vessels in the body region.

In step II, a second contrast medium application is performed in the form of a bolus larger than in the first contrast medium application, In this bolus, the contrast medium K is administered, for example, at more than 4 ml/s and is intended to lead to an intense and rapid increase in the contrast medium concentration KK in the vessels.

This is usually not desirable, as it can lead to errors in the recordings. However, an equilibrium concentration has already been established by the first contrast medium application, whereby these errors are avoided.

In step III, spectral CT data D is recorded at at least two different beam energies. This can be performed, for example, with a gantry 2 of a CT system 1 according to FIG. 1.

In step IV, images Z, E are reconstructed from the CT data D. In this example, segmentation of different base materials is to take place in the reconstruction of the images Z, E, and, depending on the concentration, segments in the images Z, E are to be selectively highlighted. For example, a VNC image is reconstructed as an intermediate image Z and a material image is reconstructed as a further intermediate image Z. The VNC image simulates a CT image without contrast medium and the material image represents information of local concentrations of the contrast medium. The result image E is then created from these intermediate images Z by image superimposition, for example by subtraction methods.

The reconstructed images Z, E are then output. In this case, only the result image E can be output or also the intermediate images Z.

Reference is again made to the fact that the figures that are described above in detail are only exemplary embodiments that can be modified in various ways by the person skilled in the art without departing from the scope of the present invention. Furthermore, the use of the indefinite article “a” or “an” does not rule out that the relevant features can also be provided multiple times. Likewise, the terms “unit” and “device” do not rule out that the relevant components are made of multiple interacting part components that where necessary can also be spatially distributed. The expression “a number” is to be understood as “at least one”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Independent of the grammatical term usage, individuals with male, female or other gender identities are included within the term.

What is claimed is:

1. A method of operating an X-ray device for creating reconstructed images of a body region, the method comprising:
triggering administration of a first contrast medium application for presaturation of the body region with contrast medium, the first contrast medium application being administered to achieve a desired equilibrium concentration;
triggering administration of a second contrast medium application in the form of a bolus larger than in the first contrast medium application;
recording spectral image data at at least two different beam energies;
reconstructing images from the spectral image data; and
outputting the images.

2. The method as claimed in claim 1, wherein, in the bolus of the second contrast medium application, the contrast medium is administered at more than 4 ml/s.

3. The method as claimed in claim 1, wherein the reconstructing of the images comprises:
segmenting different base materials; and
selectively highlighting, attenuating or masking segments in an image depending on the concentration, wherein at least one of
areas having increased contrast medium concentrations are selectively enhanced,
contrast medium concentrations lying outside a value range are visualized on virtual native images,
contrast having concentrations that are below a first threshold value are selectively enhanced, wherein
a detection of fresh versus organized thrombi is included for the selective enhancement,
areas with a concentration above a second threshold value are masked, not further amplified or linearly co-amplified,
arterial and venous phases in the brain or bronchus are separated, or
a distinction is made between an arterial and portal venous phase of an accumulation of contrast medium in the liver.

4. The method as claimed in claim 1, further comprising:
reconstructing a VNC image and a material image, wherein
the VNC image simulates an image recording without contrast medium, and
the material image represents information of local concentrations of the contrast medium; and
creating a result image from the VNC image and the material image by at least one of image superimposition or masking image information that lies outside a threshold value range, the image superimposition including subtraction methods.

5. The method as claimed in claim 1, wherein at least the first contrast medium application is performed by an automatically controlled application unit, wherein a contrast medium concentration has been selected for the second contrast medium application and the contrast medium concentration is diluted for the first contrast medium application.

6. The method as claimed in claim 1, further comprising:
determining a point in time of the second contrast medium application using the first contrast medium application.

7. The method as claimed in claim 1, wherein
the contrast medium is administered at more than 6 ml/s in the second contrast medium application,
the second contrast medium application is adapted to at least one of cardiac output, anatomy to be analyzed, or pathology, and
the bolus in the second contrast medium application is equivalent in behavior to a conventional contrast medium application.

8. The method as claimed in claim 1, wherein the X-ray device is a computed tomography device, and the method is combined with information from spectral topograms for planning a CT image by automated detection of a pathological aorta with a protocol proposal and placement of a region of interest (ROI) to determine a cardiac output so as to at least one of determine a second bolus phase or estimate a soft tissue compartment, including determination of fatty tissue to parenchyma ratio, and based on this, determination of a necessary presaturation.

9. The method as claimed in claim 1, wherein at least one of limits of value ranges, threshold values, applied contrast medium quantities, contrast medium concentrations or kernels for reconstruction of images are selected based on at least one of a tumor biology, a selected examination type or a body region to be examined.

10. The method as claimed in claim 1, further comprising:
performing additional recordings of image data at least one of between the first contrast medium application and the second contrast medium application or at different recording points in time after the second contrast medium application, the additional recordings of image data being performed at at least two different beam energies; and
creating a result image for different recording points in time from respective recorded images by at least one of image superimposition or by masking image information that lies outside a threshold value range, the image superimposition including subtraction methods.

11. A system comprising:
an application unit configured to
trigger administration of a first contrast medium application for presaturation of a body region with contrast medium, the first contrast medium application being administered to achieve a desired equilibrium concentration,
trigger administration of a second contrast medium application in the form of a bolus larger than in the first contrast medium application, wherein in the bolus the contrast medium is administered at more than 4 ml/s;
a number of X-ray device components configured to record spectral image data at at least two different beam energies;
a reconstruction unit configured to reconstruct images from the spectral image data, wherein
in reconstructing the images, concentration-dependent portions in the image are selectively enhanced, attenuated or masked; and
an output unit configured to output the images.

12. The system as claimed in claim 11, wherein the application unit is configured to
automatically dilute the contrast medium for the first contrast medium application to, or by, a defined first value,
apply the contrast medium for the first contrast medium application in a first diluted manner,
automatically dilute the contrast medium for the second contrast medium application to, or by, a defined second value, and
apply the contrast medium for the second contrast medium application in a second diluted manner.

13. A control device configured to control a computed tomography system, the computed tomography system configured to operate according to the method as claimed in claim 1.

14. A computed tomography system comprising the system as claimed in claim 11.

15. A non-transitory computer program product comprising commands that, when executed by a computer at a system, trigger the system to operate according to the method as claimed in claim 1.

16. A non-transitory computer-readable medium storing computer-executable instructions that, when executed by a computer at a system, cause the system to operate according to the method as claimed in claim 1.

17. The method as claimed in claim 1, wherein the reconstructing of the images comprises:
segmenting different base materials; and
selectively highlighting, attenuating or masking segments in an image depending on the concentration.

18. The method as claimed in claim 1, further comprising:
reconstructing a VNC image and a material image, wherein
the VNC image simulates an image recording without contrast medium, and
the material image represents information of local concentrations of the contrast medium; and
creating a result image from the VNC image and the material image by at least one of image superimposition or masking image information that lies outside a threshold value range.

19. The method as claimed in claim 1, wherein
the contrast medium is administered at more than 5 ml/s in the second contrast medium application,
the second contrast medium application is adapted to at least one of cardiac output, anatomy to be analyzed, or pathology, and
the bolus in the second contrast medium application is equivalent in behavior to a conventional contrast medium application.

20. The system as claimed in claim 11, wherein the application unit is configured to
automatically dilute the contrast medium for the first contrast medium application to, or by, a defined first value,
apply the contrast medium for the first contrast medium application in a first diluted manner, and
apply the contrast medium for the second contrast medium application in an undiluted manner.
